(19) United States
(12) Patent Application Publication
(10) Pub. No.: US 2006/0258534 A1
(43) Pub. Date: Nov. 16, 2006

(54) Rhizobium leguminosarum strain and use thereof as plant inoculant

(76) Inventors: James Darren Hill, Saskatoon (CA); Mary Elizabeth Leggett, Saskatoon (CA)

Correspondence Address: KIRBY EADES GALE BAKER, BOX 3432, STATION D, OTTAWA, ON, K1P 6N9, CA

(21) Appl. No.: 11/129,317

(22) Filed: May 16, 2005

(51) Int. Cl. A01N 25/26; C12N 1/20

(52) U.S. Cl. 504/100; 435/252.2

(57) ABSTRACT

A novel strain of Rhizobium leguminosarum designated S012A-2 (IDAC 080305-01). The strain is useful for improving plant growth and yield of legumes, particularly peas and lentils by nitrogen fixation. The strain is contacted with legume seeds prior to and/or during germination and growth, and may be used to form an inoculant composition that can be used to coat seeds prior to sowing or added to furrows during planting.

### BACKGROUND OF THE INVENTION

I. Field of the Invention

This invention relates to inoculants used to promote plant growth and yield. More particularly, the invention relates to inoculants of this kind containing strains of Rhizobia used with legumes, e.g. peas and lentils, for improving nitrogen fixation, nodulation, etc.

II. Description of the Prior Art

Biological nitrogen fixation is the consequence of a complex and unique symbiosis between Rhizobium bacteria and legume host plants. The first stage in this process is the formation of nodules which occurs by the penetration of the host root hairs by rhizobial bacteria, followed by the formation of a rhizobial infection thread which moves into the host plant's root cortex, after which the rhizobial bacteria are encased in specialized plant cells and then undergo rapid multiplication. Subsequently, the rhizobial bacteria become pleomorphic, their nuclear material degenerates and the resulting bacteroids develop the enzyme complexes, particularly nitrogenase, required for nitrogen fixation (Paul, E. A. and F. E. Clark, 1989, Soil Microbiology and Biochemistry. Academic Press Inc. San Diego. pp. 182-192). The environmental, nutritional and physiological conditions required for rhizobial cell growth and the successful establishment of efficient nitrogen-fixing symbioses are known (Trinick, M. J., 1982, IN W. J. Broughton (Ed.), Nitrogen Fixation Vol. 2, Clarendon Press, Oxford. pp. 76-146).

The amounts of nitrogen fixed by legume:Rhizobium symbioses are significant and, in agricultural situations, can be used to supplement or replace nitrogen fertilizer applications. For example, a typical rate of nitrogen fixation by nodulated alfalfa is up to 250 kg/hectare/year (Atlas, R. M. and R. Bartha, 1981, Microbial Ecology: Fundamentals and Applications, Addison-Wesley Pub. Co. Reading. pp. 364-365) and up to 450 kg/ha/yr by nodulated soybeans (Peoples, M. B. and E. T. Craswell, 1992, Plant Soil 141: 13-39). Consequently, legume crops have become an integral component of most field crop rotations used in agriculture around the world.

Commercial rhizobial inoculant compositions are commonly used when planting legume crops to ensure that sufficient rhizobial bacteria are present to establish effective nitrogen-fixing systems. Various types of commercial Rhizobium inoculant carriers, compositions and preparations are known including liquids, powders and granules (Thompson, J. A., 1991, IN Report of the Expert Consulation on Legume Inoculant Production and Quality Control (J. A. Thompson, Ed.) Food and Agriculture Association of the United Nations, Rome, pp. 15-32).

Even though such rhizobial inoculant compositions are already known, there is always a desire to find and utilize improved versions that are more effective or advantageous, at least for specific crops and growth environments.

### SUMMARY OF THE INVENTION

An object of the present invention is to enable legume crops to fix nitrogen at high rates in order to generate good crop growth and/or yields.

The present invention provides a novel strain of the bacterium Rhizobium leguminosarum (designated strain SO12A-2) in isolated and/or purified form that can be used to inoculate legume plants to improve growth and yield by nitrogen fixation.

The invention also relates to inoculant compositions containing the novel strain, to seeds coated with the inoculant compositions, and to methods of improving plant growth and yield employing the novel strain.

An advantage of the invention, at least in preferred forms, is that it can improve the property of Rhizobium leguminosarum for assisting legumes in the fixing of nitrogen for use by the plants, e.g. by increasing nodulation, thereby improving nitrogen fixation, plant growth and productivity in legumes.

Deposit of Microorganisms

Isolated and purified (microbially pure) samples of strain SO12A-2 of Rhizobium leguminosarum as disclosed herein were deposited at the INTERNATIONAL DEPOSITARY AUTHORITY OF CANADA (IDAC) of 1015 Arlington Street, Winnipeg, Manitoba, R3E 3R2, Canada (Telephone: (204) 789-2070; Facsimile: (204) 789-2097) for patent purposes under the terms of the Budapest Treaty. The deposit was made on Mar. 8^{th}, 2005 and the deposit receipt number is IDAC 080305-01.

Definitions

Colony forming unit (cfu): The minimum number of bacteria that, when assembled together as a propagation unit, can be grown and propagated successfully on agar medium under favorable conditions.

Increased growth and/or yield: The increases are in comparison to growth and/or yield of an identical legume crop grown under identical conditions (and preferably at the same time in immediately adjacent areas) from uninoculated seed, or (when compared with known inoculants) grown from seed inoculated with a known commercial species of Rhizobium leguminosarum, and generally the species identified herein as PBI #108. The plant growth and yield values are the averages of a statistically significant numbers of plants taken from each plant crop and compared directly.

### DETAILED DESCRIPTION OF THE INVENTION

As noted above, the present invention relates primarily to a novel strain of Rhizobium leguminosarum bacteria (a strain designated by the applicants herein as strain SO012A-2 and deposited at an international patent depository as indicated above) and its use to improve growth and productivity of legume crops, particularly pea and lentil, by enhancement of nitrogen fixation by the growing plants.

The new strain SO12A-2 is one of several isolated from the natural environment as described in the Experimental Details section below and found to be superior for enhancing legume plant growth and yield.

The novel strain was obtained from the following location:

Collection Site Information

| | Location: | | | | |
|---|---|---|---|---|---|
| Sample | (Nearest Town | Latitude | Date | Description of | Plant |
| ID# | or City) | Longitude | Collected | Surroundings | Association |
| S012A-2 | Battleford, SK | 52° 48′ 47 N | Aug. 9, | Aspen bluff, | Lathyrus |
| | | 108° 29′ 29 W | 2001 | located in | venosus |
| | | | | sandhills, | |
| | | | | mixed grasses. | |
| | | | | Aspen have | |
| | | | | white trunks | |

The novel strain can be propagated from a small sample by conventional methods of bacterial growth and multiplication. In the present invention, isolated and pure samples of the strain SO12A-2 multiplied in this way are normally used to prepare an inoculant composition by infecting a preferably sterile inoculant carrier with the bacterial strain. The inoculant composition is then used to inoculate a legume crop, preferably by planting seeds of the crop in contact with the inoculant composition, ideally by coating the seeds with the inoculant composition (peat or a liquid, for example) prior to planting. Alternatively, a granular or liquid product that contains the specific strain can be added directly to the soil (e.g. in soil furrows). The seed are then planted in the furrow with the soil applied inoculant.

When legume seeds are contacted with an inoculant composition, successful inoculation of the seeds with the bacterial strain, and the resulting benefits to the legume:Rhizobium symbiosis, are not limited to a particular inoculant carrier type, a particular inoculation process, or a particular legume:Rhizobium symbiosis, but rather, can be accomplished in a variety of ways. The carrier employed may be liquid or solid (e.g. a powder or granules—i.e. aggregates consisting of particles bound together), but the preferred inoculant carrier is an organic solid, for example peat. Seeds may be coated with an aqueous slurry of sterilized peat infected with the bacterial strain and then allowed to dry. Alternatively, the seeds may be directly dry-coated with infected powdered peat having a moisture content of, for example, 6 to 20% by weight. Most preferably in such cases, the powdered peat (or other solid inoculant carrier) contains a sticking agent that facilitates the adhesion of the inoculant composition to the legume seeds. Examples of suitable sticking agents include alginate, graphite, gum arabic and methyl cellulose used in quantities sufficient to ensure the required adhesion to the seeds.

In the case of liquid formulations, there are many potential ingredients for producing such formulations. Possible liquid formulants include: water, glycerol, polymers (polyvinyl alcohol or polyvinyl pyrrolidone, for example), glucose, yeast extract, NaOH and buffers (KH₂PO₄, for example).

An example of a method of forming a liquid inoculant composition is to obtain an aliquot of novel Rhizobium cells from a stock culture. This aliquot is inoculated aseptically into a culture medium containing a carbon source, yeast autolysate and buffering components. The culture is then incubated for 4-8 days at 30° C. with shaking. Subsequently, formulation components are added to the culture medium. The formulated liquid culture is then transferred aseptically into previously gamma-irradiated 5 L polyethylene bags and stored at room temperature. The final titre of the bags is in the range of 1×10⁶ to 1×10¹¹ cells per mL.

An example of a method of forming a granular inoculant composition is to obtain an adequate granule source (peat, clay or gypsum for example). This granule is then mixed with a volume of novel Rhizobium culture which was grown for 4-8 days at 30° C. in a medium containing a carbon source, yeast autolysate and buffering components. The culture is added to the carrier at such a rate as to yield a final moisture content of 30% wet weight. Other formulants are also added at this time. The formulation is mixed to a uniform consistency, transferred to 20 Kg polyethylene lined paper bags and left to cure at 25° C. for 1 to 3 weeks. The final titer of the bags is in the range of 1×10⁶ to 1×10¹¹ cfu/gram.

An example of a way of forming a inoculant composition containing peat is to package peat (having a moisture content of preferably 6 to 20% by weight) in plastic bags of an appropriate size for sale and use, with or without a sticking agent, and then to sterilize the bags in a manner that ensures complete absence of contaminating microorganisms. Using aseptic techniques, an aqueous suspension of the novel Rhizobium cells is then added to each bag in a concentration appropriate to produce the preferred number of cfu/gram in the final inoculant product. The total volume of suspension added to each bag is preferably such that the final moisture content of the composition does not exceed 50% by weight. In fact, a more preferred final content is in the range of 40 to 45% by weight. After the microbial suspension has been mixed well with the peat (e.g. by massaging or tumbling the bags), the bags are cured at a temperature in the range of 20 to 30° C. for a period of 7 to 35 days prior to storage at ambient temperature. If a sticking agent is incorporated into the peat prior to sterilization, the composition can be directly applied to legume seeds or, alternatively, the seeds can be dampened prior to coating. If a sticking agent is not incorporated into the peat, the composition may be made into a slurry by adding the composition plus a sticking agent to a volume of water an mixing well before coating seeds. Examples of sticking agents used in this way include honey, skim milk and wallpaper paste, in addition to the sticking agents already mentioned above. Legume seeds coated in this way may be handled and planted in the same way as seeds coated with other materials.

Alternatively, a liquid rhizobial inoculant can be applied directly to legume seeds or applied in-furrow and a granular rhizobial inoculant can be applied in-furrow with the legume seed.

It is preferred that legumes with large-sized seeds, e.g. peas and lentils, receive a range of 1×10³ to 1×10⁷ colony forming units per seed (cfu/seed) of Rhizobium leguminosarum strain S012A-2.

Examples of preferred legume seeds that can be inoculated with Rhizobium leguminosarum strain S012A-2 include peas (Pisum spp.) and lentils (Lens culinaris).

If desired, the novel strain of Rhizobium leguminosarum of the present invention may be used in combination with Penicillium bilaii (also used is Penicillium bilaiae), a phosphate-solubilizing soil fungus as disclosed in U.S. Pat. No. 5,026,417 which issued to Reginald Kucey on Jun. 25, 1991 (the disclosure of which is incorporated herein by reference). The fungus Penicillium bilaii is a known micro-organism. A fungus identified as Penicillium bilaii was deposited at the American Type Culture Collection in Rockville, Md., USA (now moved to Manassas, Va., 20108, USA) under the deposit number ATCC 20851 (1974 edition of the ATCC catalogue). This is believed to be the same micro-organism. In any event, the name P. bilaii is used for the micro-organism throughout this specification. An inoculant containing P. bilaii can be obtained commercially under the trademark JumpStart from Philom-Bios Inc., of 318-111 Research Drive, Saskatoon, Saskatchewan, Canada. Preferred ways of combining P. bilaii with Rhizobia are disclosed in U.S. Pat. No. 5,484,464, which issued to Gleddie et al. on Jan. 16, 1996 (the disclosure of which is incorporated herein by reference).

The nodulation and nitrogen fixation processes in legume:Rhizobium symbioses require substantial energy expenditures by the plant host and, therefore, considerable soluble phosphate is required to ensure that these processes proceed at optimal rates. Since P. bilaii has the properties of solubilizing insoluble phosphate from native and applied solid forms, e.g. precipitated calcium phosphate, rock phosphate, and various types of phosphate fertilizers, the essence of the combination of P. bilaii with the novel rhizobial strain of the present invention relates to increased availability of soluble phosphate and fixed nitrogen to the legume:Rhizobium symbioses as a consequence of the P. bilaii activity, such that the rhizobial strain is better able to provide benefits to legume nitrogen fixation, plant growth and productivity.

These inoculant compositions containing P. bilaii and the novel rhizobial strain of the present invention can be formed and used without difficulty in much the same way as the inoculant compositions of the rhizobial strain itself Combination Penicillium bilaii and rhizobial inoculant compositions are available commercially under the trademark TagTeam from Philom Bios Inc., of 318-111 Research Drive, Saskatoon, Saskatchewan, Canada.

As an example, using aseptic techniques, a suspension of P. bilaii spores and Rhizobium cells may be transferred into sterilized bags of peat such that the final concentration of spores after the composition step is completed is in the range of 1×10⁴ to 1×10⁷ cfu/g, and the titre of Rhizobium cells after the composition step is completed is in the range of 1×10⁵ to 1×10¹¹ cfu/g. If a sticking agent is incorporated into a peat carrier prior to sterilization, the resulting composition can be directly applied to the appropriate legume seeds or, alternatively, the seeds can be dampened prior to the inoculation step. Legume seeds inoculated with Penicillium bilaii and rhizobial inoculant compositions are handled and planted in the same manner as legume seeds inoculated only with rhizobial inoculants.

In the operation of the present invention, after being contacted with the novel strain of Rhizobium leguminosarum (either with or without P. bilaii), the legume plants may be germinated and grown in a manner entirely identical to the germination and growth of untreated legume crops, e.g. by planting seeds and subjecting the seeds to conditions of moisture, sunlight and temperature that promote plant growth and development to maturity. Conventional fertilizers, pesticides, soil amendments, and the like, may be used in the conventional manner, if required or desirable. Conventional harvesting practices may be employed. Such operations are clearly well known to farmers and agriculturalists and require no further discussion or explanation.

The isolation and testing of the novel strain of Rhizobium leguminosarum according to the present invention is illustrated in the following Experimental Details.

### EXPERIMENTAL DETAILS

### Experiment 1

### Comparison of Eight Newly Isolated Rhizobium Strains Against Known Strains PBI #108 and PBI #101

Purpose/Background

1) To evaluate eight previously untested Rhizobium strains for their ability to enhance biomass accumulation and nitrogen in legume plant tissue. These eight strains are evaluated against known strains PBI #108 and PBI#101. Note that PBI #108 is a commercial strain of Rhizobium leguminosarum that can be obtained from the Australian Legume Inoculants Research Unit of the New South Wales Agriculture Horticultural Research & Advisory Station, Locked Bag 26, Gosford, New South Wales, 2250, Australia under the deposit number ALIRU SU303. PBI #101 is a strain available from the USDA under deposit number 2449.
   Experimental Design:

Factorial Design: 2 Soil Types (Aberdeen soil, Kyle soil)
1 Pea seed Cultivar (‘Mozart’)
12 Seed Treatments (Uninoculated, Nitrogen, Eight untested strains, PBI #108, PBI #101)
Randomized Block.
   Strain Selection Criteria:
   Eight strains varied on their size, color, and morphology. Strains were selected based on their uniqueness of these three traits and their geographic location.
   Material & Methods:
1) Collected field soil was sifted through a ¼ inch screen to remove any lumps of soil, roots, other plant material, sticks, etc. The soil was then spread out to dry for a period of one week and then placed into plastic bins until needed.
2) Pots (4½ inch×5 inch deep) were then labeled according to treatment.

A sterile square pieces of spun polyester (black landscape fabric) was then placed into the bottom of each of the pots to prevent the sand/soil mixture from draining out through the pots drainage holes.
3) A 50% mixture of Kyle soil/silica sand or 50% Aberdeen soil/silica sand (Unimum Industries-industrial quartz) was used as a potting media.
4) After filling each pot with the sand/soil mixture each pot was placed into a large plastic Ziploc™ bag.
5) One day before seeding each pot was watered with 150 ml of tap water (non-sterile) and the bags were sealed until seeded.
6) On the day of seeding 5.5 kg of pea seed-cultivar ‘Mozart’-was divided up into eleven 500 gram amounts and surfaced sterilized via the following method:
   a) Place 500 grams of seed into a 2 liter glass Erlenmeyer flask
   b) Cover the seeds with 95% Ethanol, let stand 60 seconds then drain.
   c) Add a fresh preparation of a 50% bleach solution (1000 mls into 1000 mls water-2.6% NaOCl active), add seed, shake and let stand 5 minutes, then drain.
   d) Rinse with 4 changes of R.O. water (non-sterile) followed by one rinse with sterile R.O. water.
7) After surface sterilization each 500 g amount of seed was spread into an aluminum foil pan lined with sterile paper towel and blotted dry and then transferred into clean large Ziploc™ bags.
8) After inoculation uninoculated and inoculated pea seeds were planted. Five pea seeds were placed into each pot 2 inches below the surface. Spoons and forceps used to plant the seed were washed and dried thoroughly between treatments.

9) The bags were then sealed and placed into a growth chamber that was set for the following conditions:

| | |
|---|---|
| a. Day length | 16 Hrs at 21.5° C. |
| b. Night length | 8 Hrs at 16° C. |
| c. R.H. | Not controlled |
| d. Lighting Source | Metal Halide, High Pressure Sodium |
| e. Intensity | Not measured. |

10) Each pot was placed into a plastic bin in a randomized block design (two bins contained one pot of each of the 24 treatments). A total of 20 bins were used to hold all of the treatments. Three times a week (Monday's, Wednesdays and Fridays) each plastic bin was moved one bin position to the right. This was done to ensure differences in light intensity/quality or temperature were consistent for each of the treatments inside the growth chamber.
11) Bags were kept fully closed until 50% emergence was observed, then fully opened.
12) Plants were checked daily and watered as required using tap water.
13) After 35 days plants were photographed (digital image file) and then harvested.

Results:

TABLE 1.1
Visual Assessment of Plant Color
Ranking Based on Color: Kyle Soil
Ranked from darkest green to yellow/green

| | |
|---|---|
| 1 | Nitrogen |
| 2 | S012A-2 |
| 3 | S008A-1 |
| 4 | S024B-3 |
| 5 | PBI#108 |
| 6 | S030B-1 |
| 7 | S016B-3 |
| 8 | Uninoculated |
| 9 | S017B-3 |
| 10 | S025A-5 |
| 11 | PBI#101 |
| 12 | S020B-1 |

TABLE 1.2
Ranking by mean shoot dry weight:
Kyle Soil

| | Seed | Shoot dry weights†: |
|---|---|---|
| Ranking: | Treatment: | (grams per shoot) |
| 1 | Nitrogen | 0.46a |
| 2 | PBI #108 | 0.39b |
| 3 | PBI #101 | 0.37bc |
| 4 | S024B-3 | 0.36bcd |
| 5 | S016B-3 | 0.35bcd |
| 6 | S012A-2 | 0.35bcd |
| 7 | S020B-1 | 0.33bcd |
| 8 | S030B-1 | 0.32cd |
| 9 | S008A-1 | 0.32cd |
| 10 | Uninoculated | 0.31cd |
| 11 | S017B-3 | 0.30d |
| 12 | S025A-5 | 0.29d |

†Means followed by a different letter are significantly different at p = 0.05

TABLE 1.3
Ranking by mean shoot dry weight:
Aberdeen Soil

| | Seed | Shoot dry weights†: |
|---|---|---|
| Ranking: | Treatment: | (grams per shoot) |
| 1 | S024B-3 | 0.49a |
| 2 | S025A-5 | 0.47ab |
| 3 | Nitrogen | 0.45abc |
| 4 | S008A-1 | 0.43abcd |
| 5 | Uninoculated | 0.43abcd |
| 6 | PBI #101 | 0.41abcde |
| 7 | S012A-2 | 0.38bcde |
| 8 | S017B-3 | 0.37cde |
| 9 | S030B-1 | 0.36de |
| 10 | PBI #108 | 0.35de |
| 11 | S020B-1 | 0.35de |
| 12 | S016B-3 | 0.33e |

†Means followed by a different letter are significantly different at p = 0.05
†Means followed by a different letter are significantly different at p = 0.05
‡Two replicates per treatment. Replicates 1to 5 and 6 to 10 were combined prior to analysis

TABLE 1.4
Ranking by % Nitrogen:
Kyle Soil

| | Seed | |
|---|---|---|
| Ranking: | Treatment: | % Nitrogen†‡ |
| 1 | S012A-2 | 3.33a |
| 2 | S008A-1 | 3.17ab |
| 3 | S030B-1 | 2.87abc |
| 4 | Nitrogen | 2.80bc |
| 5 | PBI#108 | 2.79bc |
| 6 | S024B-3 | 2.59cd |
| 7 | S016B-3 | 2.23de |
| 8 | PBI#101 | 2.14def |
| 9 | S017B-3 | 1.81efg |
| 10 | Uninoculated | 1.78efg |
| 11 | S025A-5 | 1.76fg |
| 12 | S020B-1 | 1.60g |

†Means followed by a different letter are significantly different at p = 0.05
‡Two replicates per treatment. Replicates 1to 5 and 6 to 10 were combined prior to analysis

TABLE 1.5
Ranking by Total Nitrogen per Shoot
Kyle

| | | Total Nitrogen |
|---|---|---|
| Ranking: | Seed Treatment: | (mg/shoot) |
| 1 | Nitrogen | 0.0131a |
| 2 | S012A-2 | 0.0117ab |
| 3 | PBI#108 | 0.0.09abc |
| 4 | S008A-1 | 0.0101bcd |
| 5 | S030B-1 | 0.0093bcd |
| 6 | S016B-3 | 0.0086cd |
| 7 | S024B-3 | 0.0082cde |
| 8 | PBI#101 | 0.0080de |
| 9 | Uninoculated | 0.0059ef |
| 10 | S017B-3 | 0.0054f |
| 11 | S020B-1 | 0.0053f |
| 12 | S025A-5 | 0.0050f |

TABLE 1.6
Ranking by % Nitrogen:
Aberdeen Soil

| | Seed | |
|---|---|---|
| Ranking: | Treatment: | % Nitrogen†‡ |
| 1 | S012A-2 | 3.23a |
| 2 | Nitrogen | 3.18a |
| 3 | S016B-3 | 3.15a |
| 4 | S030B-1 | 3.11ab |
| 5 | S008A-1 | 3.07ab |
| 6 | S024B-3 | 3.03ab |
| 7 | PBI#108 | 2.99abc |
| 8 | Uninoculated | 2.86abc |
| 9 | S017B-3 | 2.77bc |
| 10 | S025A-5 | 2.66c |
| 11 | PBI#101 | 2.64c |
| 12 | S020B-1 | 2.64c |

TABLE 1.7
Ranking by Total Nitrogen per Shoot
Aberdeen

| | | Total Nitrogen |
|---|---|---|
| Ranking: | Seed Treatment: | (mg/shoot) |
| 1 | S024B-3 | 0.0152a |
| 2 | Nitrogen | 0.0144a |
| 3 | S008A-1 | 0.0132ab |
| 4 | S012A-2 | 0.0124abc |
| 5 | Uninoculated | 0.0123abc |
| 6 | S016B-3 | 0.0110bcd |
| 7 | S030B-1 | 0.0110bcd |
| 8 | PBI#101 | 0.0108bcd |
| 9 | PBI#108 | 0.0105bcd |
| 10 | S017B-3 | 0.0104bcd |
| 11 | S025A-5 | 0.0099cd |
| 12 | S020B-1 | 0.0092d |

### Experiment 2

### Field Trials

Various field trials were performed over a three year period to assess the ability of Rhizobium leguminosarum strain S012A-2 to enhance seed yield as compared to a commercial inoculant strain (PBI#108). Field protocols are outlined below.

### Trial: Pea 2002 and 2003

Seeding Guidelines:

Reps: 6

Variety: Mozart

Fertilizer: 20 kg P₂O₅ ha⁻¹ side banded for all treatments.

Seeding rate: 350,000 plants ac⁻¹=88 plants m²=3.5 bu ac⁻¹

Seed treatment: Apron

Row spacing: 8 inch

Equipment: Air seeder, stealth openers, fertilizer one inch to the side and below seed.

Product: All strains were formulated in a peat carrier and applied at 2.2 kg/1320 kg seed. The minimum guarantee was 7.4×10⁸ Rhizobium leguminosarum strain S012A-2 cells per gram.

### Trial: Lentil 2002, 2003 and 2004

Seeding Guidelines:

Reps: 6

Variety: Grandora

Fertilizer: 20 kg P₂O₅ ha⁻¹ side banded for all treatments.

Seeding rate: 530,000 plants ac⁻¹=111 kg ha⁻¹=1.7 bu ac⁻¹

Seedtreatment: Apron FL and

Row spacing: 8 inch

Equipment: Air seeder, stealth openers, fertilizer one inch to the side and below seed.

Products: All strains were formulated in a peat carrier and applied at 2.2 kg/820 kg seed. The minimum guarantee was 7.4×10⁸ Rhizobium leguminosarum strain S012A-2 cells per gram.

TABLE 2.1
Combined Year Yield Data for PBI #108 vs S012A-2 (Pea)

| | | Strain PBI#108 yield | Strain S012A-2 yield |
|---|---|---|---|
| Year | Location | (kg ha⁻¹) | (kg ha⁻¹) |
| 2002 | Cadillac | 3119 | 2819 |
| 2002 | Wymark | 3479 | 3483 |
| 2003 | Moon Lake | 1705 | 1765 |
| 2003 | Aberdeen | 1616 | 1613 |
| 2003 | Langham | 3351 | 3788 |
| 2003 | St. Louis | 2347 | 2238 |
| Average | | 2603 | 2618 |

TABLE 2.2
Combined Year Yield Data for PBI #108 vs S012A-2 (Lentil)

| | | Strain PBI#108 yield | Strain S012A-2 yield |
|---|---|---|---|
| Year | Location | (kg ha⁻¹) | (kg ha⁻¹) |
| 2002 | Cadillac | 1868 | 2338 |
| 2002 | Wymark | 2305 | 2357 |
| 2003 | Conquest | 1375 | 1557 |
| 2003 | Moon Lake | 1790 | 1958 |
| 2004 | Aberdeen | 1526 | 1680 |
| 2004 | Langham | 3726 | 3745 |
| Average | | 2098 | 2273 |

1. An isolated strain of Rhizobium leguminosarum designated SO12A-2 (IDAC 080305-01).

2. An inoculant composition for inoculating legume seeds and germinants, containing a carrier and a strain of Rhizobium leguminosarum designated SO12A-2 (IDAC 080305-01).

3. The composition of claim 2, wherein the carrier is selected from a solid and a liquid.

4. The composition of claim 2, wherein the carrier is a solid.

5. The composition of claim 2, wherein the carrier is peat and which has a titre of Rhizobium leguminsarum strain S012A-2 cells in the range of 1×10⁵ to 1×10¹¹ cfu/gram

6. The composition of claim 2, containing a sticking agent to facilitate adherence of the composition to legume seeds.

7. The composition of claim 2, containing spores of Penicillium bilaii.

8. The composition of claim 2, wherein the carrier is a liquid and which has a titre of Rhizobium leguminsarum strain S012A-2 in the range of 1×10⁶ to 1×10¹¹ cells per mL.

9. The compostion of claim 2, wherein the carrier is a granule and which has a titre of Rhizobium leguminsarum strain S012A-2 in the range of 1×10⁶ to 1×10¹¹ cfu/gram.

10. A method of growing a legume crop which comprises inoculating seeds of the crop with a strain of Rhizobium leguminosarum designated SO12A-2 (IDAC 080305-01) prior to or during germination and growth of the seeds.

11. The method of claim 10, wherein the legume crop is pea.

12. The method of claim 11, wherein seeds of said pea crop receive 1×10³ to 1×10⁷ cfu/seed of said Rhizobium leguminosarum strain S012A-2.

13. The method of claim 10, wherein the legume crop is lentil.

14. The method of claim 13, wherein seeds of said lentil crop receive 1×10³ to 1×10⁷ colony forming units per seed of said Rhizobium leguminosarum strain S012A-2,

15. A method of increasing the growth and yield of lentils, which comprises contacting seeds or germinants of lentils with a strain of Rhizobium leguminosarum designated SO12A-2 (IDAC 080305-01) and growing said seeds or germinants into mature lentil plants.

16. A method of increasing the growth and yield of peas, which comprises contacting seeds or germinants of peas with a strain of Rhizobium leguminosarum designated SO12A-2 (IDAC 080305-01) and growing said seeds or germinants into mature pea plants.
